(19) United States
(12) Patent Application Publication
(10) Pub. No.: US 2011/0178442 A1
(43) Pub. Date: Jul. 21, 2011

(54) PATIENT FEEDBACK FOR CONTROL OF ULTRASOUND DEEP-BRAIN NEUROMODULATION

(76) Inventor: David J. Mishelevich, Playa del Rey, CA (US)

(21) Appl. No.: 13/007,626

(22) Filed: Jan. 15, 2011

Related U.S. Application Data
(60) Provisional application No. 61/295,760, filed on Jan. 18, 2010.

(51) Int. Cl. A61N 7/00

(52) U.S. Cl. 601/2

(57) ABSTRACT

Disclosed are methods and systems and methods for patient-feedback control of non-invasive deep brain or superficial neuromodulation using sound impacting one or multiple points in a neural circuit to produce acute effects and, with application in multiple sessions, Long-Term Potentiation (LTP) or Long-Term Depression (LTD) to treat indications such as neurologic and psychiatric conditions. One or more of sonic transducer positioning, intensity, frequency, dynamic sweeps, phase/intensity relationships, and firing patterns are changed through feedback from the patient to optimize patient experience through up-regulation or down regulation. Examples are decreases in acute pain or tremor due to more effective impact on the neural targets.

### CROSS REFERENCE TO RELATED APPLICATIONS

This patent application claims priority to provisional patent applications Application No. 61/295,760, filed Jan. 18, 2010 entitled “PATIENT FEEDBACK FOR CONTROL OF ULTRASOUND FOR DEEP-BRAIN NEUROMODULATION.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

Described herein are systems and methods for control of Ultrasonic Stimulation including one or a plurality ultrasound sources for neuromodulation of target deep brain regions to up-regulate or down-regulated neural activity.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. Neural structures are usually assembled in circuits. For example, nuclei and tracts connecting them make up a circuit. The potential application of ultrasonic therapy of deep-brain structures has been suggested previously (Gavrilov LR, Tsirulnikov EM, and IA Davies, “Application of focused ultrasound for the stimulation of neural structures,” Ultrasound Med Biol. 1996;22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels, which resulted in the generation of action potentials. Their stimulation is described at Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. High frequencies (defined as being in the range of 500 Hz to 5 MHz is excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

An alternative approach is described by Deisseroth and Schneider (U.S. patent application Ser. No. 12/263,026 published as US 2009/0112133 A1, Apr. 30, 2009) in which modification of neural transmission patterns between neural structures and/or regions is described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and FA Jolesz, “Demonstration of potential noninvasive ultrasound brain therapy through an intact skull,” Ultrasound Med Biol, 1998 Feb;24(2):275-83 and Clement GT, Hynynen K (2002) A non-invasive method for focusing ultrasound through the human skull. Phys Med Biol 47: 1219-1236.). Ultrasound can be focused to 0.5 to 2 mm as TMS to 1 cm at best.

### SUMMARY OF THE INVENTION

It is the purpose of this invention to provide methods and systems and methods for patient feedback control of non-invasive deep brain or superficial neuromodulation using ultrasound impacting one or multiple points in a neural circuit to produce acute effects and, with application in multiple sessions, Long-Term Potentiation (LTP) or Long-Term Depression (LTD). One or more of ultrasound transducer positioning, frequency, intensity, and phase/intensity relationships are changed through feedback from the patient to optimize the patient experience through up-regulation or down regulation. Examples are decreases in acute pain or tremor due to more effective impact on the neural targets.

### BRIEF DESCRIPTION OF THE DRAWINGS

FIG. 1 shows a control mechanism in which the patient controls delivery parameters to optimize delivery impact.

FIG. 2 illustrates a set of neural targets that are to be down-regulated using ultrasound neuromodulation under patient-feedback control to adjust acute pain.

FIG. 3 shows a block diagram of the feedback control algorithm.

### DETAILED DESCRIPTION OF THE INVENTION

It is the purpose of this invention to provide methods and systems for the adjustment of deep brain or superficial neuromodulation using ultrasound or other non-invasive modalities to impact one or multiple points in a neural circuit under patient-feedback control.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), Deep Brain Stimulation (DBS) using implanted electrodes, implanted optical stimulation, stereotactic radiosurgery, Radio-Frequency (RF) stimulation, vagus nerve stimulation, other local stimulation, or functional stimulation).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency. Spot size being smallest is not necessarily the most advantageous; what is optimal depends on the shape of the target neural structure. Such vendors as Keramos-Etalon and Blatek in the U.S., and Imasonic in France can supply suitable ultrasound transducers.

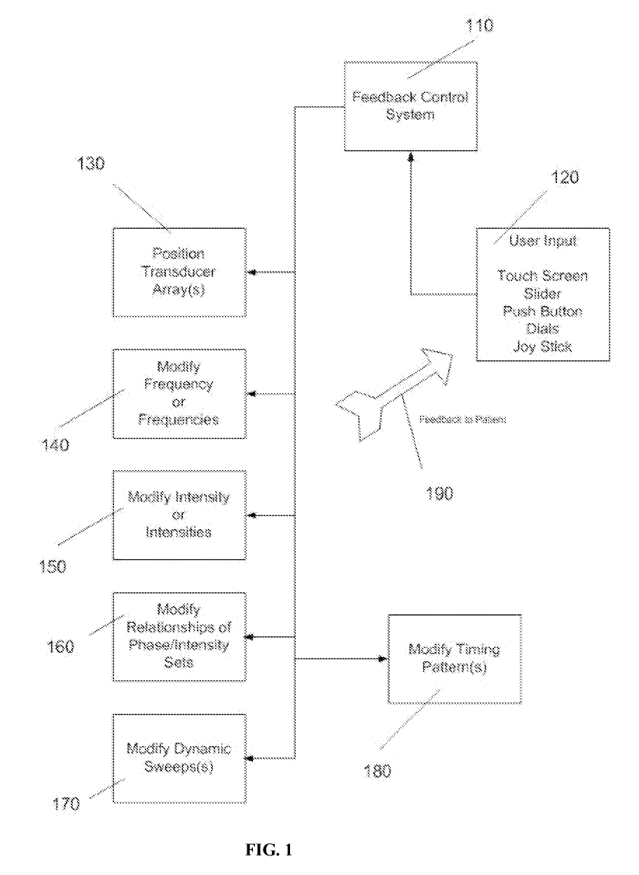

FIG. 1 shows the basic feedback circuit. Feedback Control System 110 receives its input from User Input 120 and provides control output for positioning ultrasound transducer arrays 130, modifying pulse frequency or frequencies 140, modifying intensity or intensities 150, modifying relationships of phase/intensity sets 160 for focusing including spot positioning via beam steering, modifying dynamic sweep patterns 170, and or modifying timing patterns 180. Feedback to the patient 190 occurs with what is the physiological effect on the patient (for example increase or decrease in pain or decrease or increase on tremor. User Input 120 can be provided via a touch screen, slider, dials, joystick, or other suitable means.

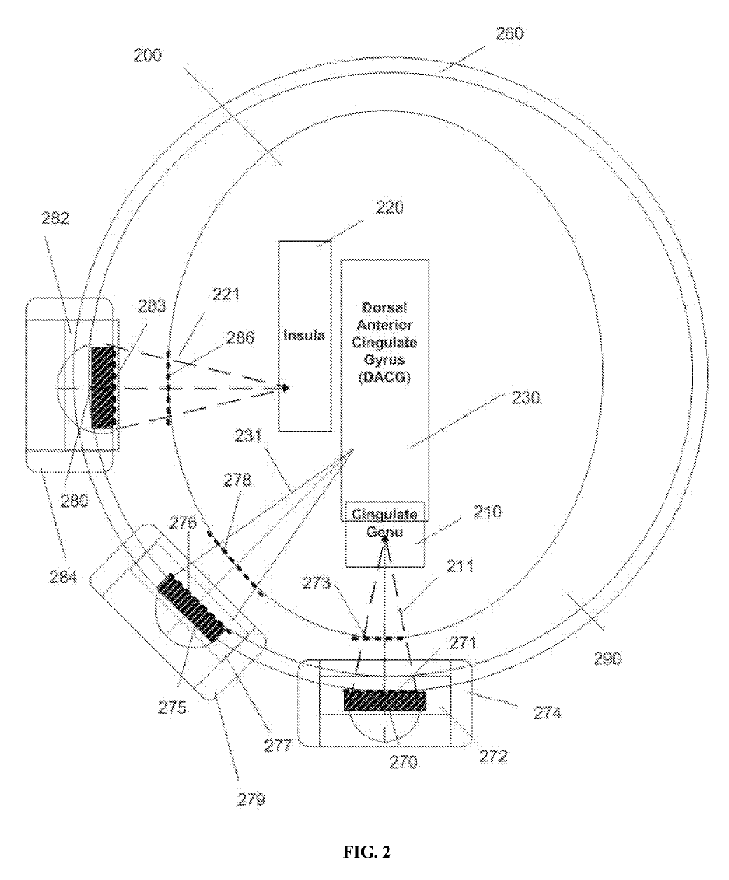

An example of a multi-target neural circuit related to the processing of pain sensation is shown in FIG. 2. Surrounding patient head 200 is ultrasound conduction medium 290, and ultrasound-transducer holding frame 260. Attached to frame 260 are transducer holders 274, 279, 284. These are oriented towards neural targets respectively holder 274 towards the Cingulate Genu 210, holder 279 towards the Dorsal Anterior Cingulate Gyms (DACG) 230, and holder 284 towards Insula 220. The assembly targeting Cingulate Genu 210, includes transducer holder 274 containing transducer 270 mounted on support 272 (possibly moved in and out via a motor (not shown)) with ultrasound field 211 transmitted though ultrasound conducting gel layer 271, ultrasound conducting medium 290 and conducting gel layer 273 against the exterior of the head 200. Examples of sound-conduction media are Dermasol from California Medical Innovations or silicone oil in a containment pouch.

The assembly targeting Dorsal Anterior Cingulate Gyms 230, includes transducer holder 279 containing transducer 275 mounted on support 277 (possibly moved in and out via a motor (not shown)) with ultrasound field 231 transmitted though ultrasound conducting gel layer 276, ultrasound conducting medium 290 and conducting gel layer 278 against the exterior of the head 200.

The assembly targeting Insula 220, includes transducer holder 284 containing transducer 280 mounted on support 282 (possibly moved in and out via a motor (not shown)) with ultrasound field 221 transmitted though ultrasound conducting gel layer 283, ultrasound conducting medium 290 and conducting gel layer 286 against the exterior of the head 200.

The locations and orientations of the holders 274, 279, 284 can be calculated by locating the applicable targets relative to atlases of brain structure such as the Tailarach atlas or via imaging (e.g., fMRI or PET) of the specific patient.

The invention can be applied to a number of conditions including, but not limited to, pain, Parkinson's Disease, depression, bipolar disorder, tinnitus, addiction, OCD, Tourette's Syndrome, ticks, cognitive enhancement, hedonic stimulation, diagnostic applications, and research functions.

One or more targets can be targeted simultaneously or sequentially. Down regulation means that the firing rate of the neural target has its firing rate decreased and thus is inhibited and up regulation means that the firing rate of the neural target has its firing rate increased and thus is excited. With reference to FIG. 2 for the treatment of pain, the Cingulate Genu 210, and DACG 230, and Insula 220 would all be down regulated. The ultrasonic firing patterns can be tailored to the response type of a target or the various targets hit within a given neural circuit.

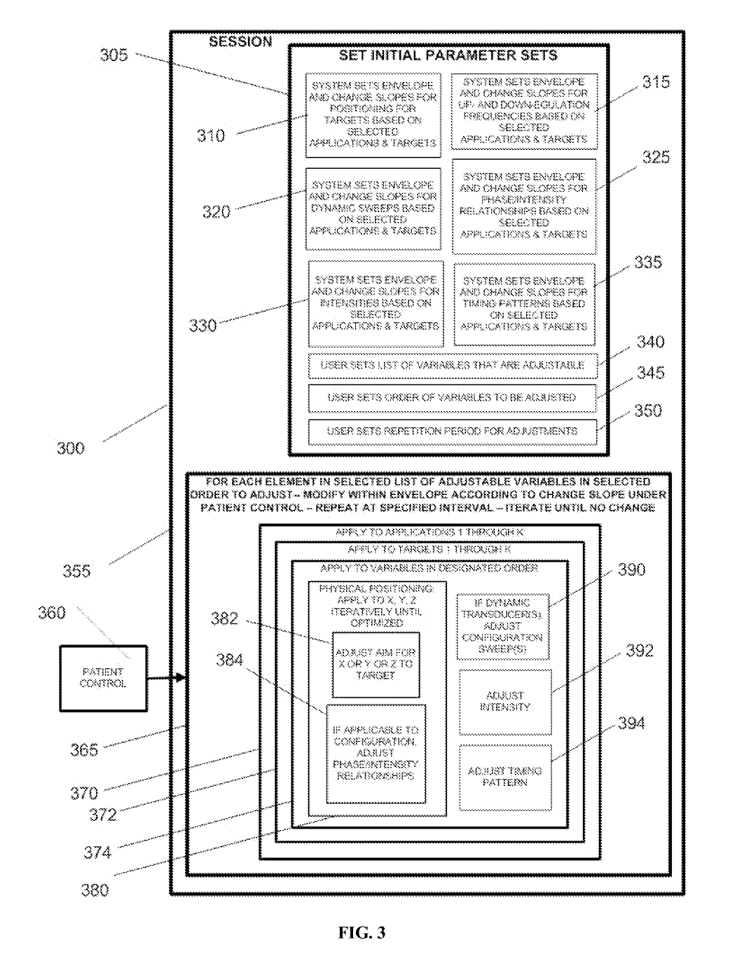

FIG. 3 shows an algorithm for processing feedback from the patient to control the ultrasound neuromodulation during a session 300. Before the real-time session begins, the initial parameters sets are set 305 by the system. This can be automatically, by the user healthcare professional instructing the system, or a combination of the two. These include setting the envelope and change slopes based on selected applications and targets for positioning for targets 310, up- and down-regulation frequencies 315, sweeps for dynamic transducers 320, phase/intensity relationships 325, intensities 330, and timing patterns 335. These are followed by the user setting what is to be controlled by the patient during the real-time feedback, namely list of variables that are adjustable 340, order of those variables to be adjusted 345, and repetition period for adjustments 350.

Once the initialization is complete the real-time part of the session begins based on patient-controlled input 360 (e.g., via touch screen, slider, dials, joy stick, or other suitable mean). During real-time processing, the outer loop 365 applies for each element in selected list of adjustable variables in selected order to adjust a modification within the envelope according to the change slope under patient control with repetition at the specified interval with iteration until there is no change felt by the patient. The process includes applying to applications 1 through k 370, applying to targets 1 through k 372, applying to variables in designated order 374, physical positioning (iteratively for x, y, z) 380 including adjusting aim towards target 382 and, if applicable to configuration, adjust phase/intensity relationships 384, in addition to adjustment of configuration sweeps if there is/are dynamic transducer(s) 390, adjust intensity 392, and adjusting timing pattern 394.

In like manner, patient-feedback control of other modalities is possible such as control of deep-brain stimulators (DBS) using implanted electrodes, Transcranial Magnetic Stimulation (TMS), transcranial Direct Current Stimulation (tDCS), implanted optical stimulation, radio-Frequency (RF) stimulation, Sphenopalatine Ganglion Stimulation, other local stimulation, or Vagus Nerve Stimulation (VNS).

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of modulating a deep-brain targets using ultrasound neuromodulation, the method comprising:
a mechanism for aiming one or a plurality of ultrasound transducers at one or more a deep-brain targets;
applying power to each of the ultrasound transducers via a control circuit thereby modulating the activity of the deep brain target region;
providing a mechanism for feedback from the patient based on the acute sensory or motor conditions of the patient; and
using that feedback to control one or more parameters to maximize the desired effect.

2. The method of claim 1, further comprising neuromodulation in a manner selected from the group of up-regulation, down-regulation.

3. The method of claim 1, wherein the means of control is orienting one or a plurality of ultrasound transducers.

4. The method of claim 1, wherein the means of control is adjusting the pulse frequency of one or a plurality of ultrasound transducers.

5. The method of claim 1, wherein the means of control is adjusting the phase/intensity relationships within and among the plurality of ultrasound transducers.

6. The method of claim 1, wherein the means of control is adjusting the intensity relationships within an ultrasound transducer or among a plurality of ultrasound transducers.

7. The method of claim 1, wherein the means of control is adjusting the fire patterns within an ultrasound transducer or among a plurality of ultrasound transducers.

8. The method of claim 1, wherein the means of control is adjusting the dynamic sweeps of a dynamic ultrasound transducer or a plurality of dynamic ultrasound transducers.

9. The method of claim 1, wherein the acoustic ultrasound frequency is in the range of 0.3 MHz to 0.8 MHz.

10. The method of claim 1, where in the power applied is less than 180 mW/cm².

11. The method of claim 1, wherein the power applied is greater than 180 mW/cm² but less than that causing tissue damage.

12. The method of claim 1, wherein a stimulation frequency for of 300 Hz or lower is applied for inhibition of neural activity.

13. The method of claim 1, wherein the stimulation frequency for excitation is in the range of 500 Hz to 5 MHz.

14. The method of claim 1, wherein the focus area of the pulsed ultrasound is 0.5 to 1500 mm in diameter.

15. The method of claim 1 where one effect is used as a surrogate for another effect.

16. The method of claim 15 where the first effect is acute pain and the second effect is chronic pain.

17. The method of claim 1, wherein a disorder is treated by neural neuromodulation, the method comprising modulating the activity of one target brain region or simultaneously modulating the activity of a plurality target brain regions, wherein the target brain regions are selected from the group consisting of NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

18. The method of claim 1, wherein the disorder treated is selected from the group consisting of pain, Parkinson's Disease, depression, bipolar disorder, tinnitus, addiction, OCD, Tourette's Syndrome, ticks, cognitive enhancement, hedonic stimulation, diagnostic applications, and research functions.

19. The method of claim 1, wherein Transcranial Magnetic Stimulation coils are used in place or ultrasound transducers.

20. The method of claim 1 wherein the feedback control of ultrasound transducers is combined with the application, with or without feedback control, of one or more other modalities selected from the group of deep-brain stimulators (DBS) using implanted electrodes, Transcranial Magnetic Stimulation (TMS), transcranial Direct Current Stimulation (tDCS), implanted optical stimulation, stereotactic radiosurgery, Radio-Frequency (RF) stimulation, vagus nerve stimulation, or functional stimulation.
